(19) United States
(12) Patent Application Publication
(10) Pub. No.: US 2023/0132619 A1
(43) Pub. Date: May 4, 2023

(54) A METHOD, AN APPARATUS, AN ASSEMBLY AND A SYSTEM SUITABLE FOR DETERMINING A CHARACTERISTIC PROPERTY OF A MOLECULAR INTERACTION

(71) Applicant: FIDA BIOSYSTEMS APS, Søborg (DK)

(72) Inventor: Henrik JENSEN, Søborg (DK)

(73) Assignee: FIDA BIOSYSTEMS APS, Søborg (DK)

(21) Appl. No.: 17/910,546

(22) PCT Filed: Mar. 10, 2021
(86) PCT No.: PCT/DK2021/050079

§ 371 Date: Sep. 9, 2022

(30) Foreign Application Priority Data
Mar. 11, 2020 (DK) PA 2020 70165
Jan. 29, 2021 (DK) PA 2021 70046

(51) Int. Cl. G01N 33/543; G01N 21/27; B01L 3/00

(52) U.S. Cl. CPC G01N 33/54373; G01N 21/272; B01L 3/502761; G01N 2021/1731

(57) ABSTRACT

The invention concerns a method, an assembly and a system for determining a characteristic property of a molecular interaction. The method includes providing a liquid sample including a particle capable of being in a state of equilibrium and in a state of non-equilibrium. The particle includes a marker in at least one of its state of equilibrium and state of non-equilibrium. The method further includes bringing the particle in a state of non-equilibrium by subjecting the sample to a condition jump comprising a jump in temperature and/or pressure; reading out the marker as a function of time during at least a portion of a relaxation time for said particle, and determining said characteristic property of said molecular interaction.

### TECHNICAL FIELD

The invention relates to a method for determining a characteristic property of a molecular interaction as well as an apparatus, an assembly and a system suitable for determining a characteristic property of a molecular interaction.

### BACKGROUND ART

Molecular interactions are important in diverse fields of protein folding, drug design, material science, sensors, nanotechnology, separations, and origins of life. In the medical science, as well as in the medicinal chemistry there is a large need for a fast and reliable determination of molecular interactions.

Biochemical and biophysical concepts of molecular interactions between ligands and their receptors are for example highly essential in drug discovery and/or drug design. Many drugs are small ligand molecules that interact with macromolecules. Affinity and specificity of ligand binding are properties that are used to determine the potential effect of a chemical compound or molecule.

Many methods and apparatus for performing determinations of properties of molecular interactions have been provided.

US2016011180 discloses a method for determining a biological response of a target to a soluble candidate substance comprising providing a concentration profile of a candidate substance in laminar flow and introducing a target and scanning the combined concentration profile to detect an optical signal representative of the biological response of the target to the soluble candidate substance.

US 2002/0090644 discloses a method and a device for determining the presence or concentration of sample analyte particles in a medium comprising: means for contacting a first medium containing analyte particles with a second medium containing binding particles capable of binding to the analyte particles; wherein at least one of the analyte or binding particles is capable of diffusing into the medium containing the other of the analyte or binding particles; and means for detecting the presence of diffused particles.

The device may for example comprise a T shaped flow device for having the first and second media in adjacent laminar flows.Polinkovsky, M., Gambin, Y., Banerjee, P. et al. Ultrafast cooling reveals microsecond-scale biomolecular dynamics. Nat Commun 5, 5737 (2014). https://doi.org/10.1038/ncomms6737, discloses a setup for measuring conformational changes of DNA hairpins using a microfluidic cell, wherein square waves of temperature are applied and the amplitude of changes in the conformations of DNA hairpins is measured as a function of frequency of the temperature waves. The square waves temperature is induced using an IR laser heating a microscopically small volume. Cooling of the heated region is accelerated by using a sapphire substrate having a high thermal conductivity.

Another system for studying protein folding is described in the article: The use of pressure-jump relaxation kinetics to study protein folding landscapes. Biochimica et biophysica acta 2006; 1764(3):489-96.

U.S. Pat. No. 9,310,359 discloses a method of performing a dispersion analysis using Flow Induced Dispersion Analysis (FIDA) for quantification of analytes such as e.g. antigens, toxins, nucleotides (DNA, RNA), etc. For pressure-driven flows of single substances, FIDA corresponds to Taylor Dispersions observed previously for pressure driven flows in tubes or thin capillaries.

There is still a need for new and reliable methods and apparatus for determining characteristic properties of molecular actions.

### DISCLOSURE OF INVENTION

An objective of the present invention is to provide a relatively fast and reliable method for determining a characteristic property of a molecular interaction as well as equipment for performing such determination.

In an embodiment, it is an objective to provide a relatively simple method for determining a characteristic property of a molecular interaction, which method is relatively fast and economical feasible.

In an embodiment, it is an objective to provide an apparatus, an assembly and/or a system suitable for performing a reliable determination of at least one characteristic property of a molecular interaction, which apparatus, assembly and/or system is/are preferably operating relatively fast, is/are durable and/or is relatively simple to operate.

These and other objects have been solved by the inventions or embodiments thereof as defined in the claims and as described herein below.

It has been found that the inventions or embodiments thereof have a number of additional advantages, which will be clear to the skilled person from the following description.

Molecular interactions are also known as noncovalent interactions or intermolecular and/or intramolecular interactions.

The phrase “molecular interaction” means any non-covalent interactions between molecules as well as within one or more molecules.

In an embodiment, the molecular interaction comprises liquid-liquid phase interaction leading to liquid-liquid phase separation (LLPS). LLPS is also known as aqueous two phase systems, biomolecular condensates or membrane less compartmentalization.

The term “particle” is herein used to mean any portion of matter comprising at least one molecule, such as an organic molecule or an inorganic molecule.

The particle may for example comprise an aggregate, a cluster, a complex or any combinations comprising one or more of these. The term “particle” includes a plurality of equal or different molecules, such as molecules of a liquid mixture, which after the condition jump may undergo a liquid-liquid phase separation.

The term “binding partner” is herein used to mean any molecule or group of molecules, capable of non-covalent interacting with the particle.

The term “marker” is herein used to mean any intrinsic or extrinsic marker capable of being detected by a reader arrangement. In an embodiment, the marker comprises an element, group of elements, moieties and/or any combination comprising one or more of these, where the marker is capable of being detected by a reader arrangement directly and/or after being influenced from an external and/or internal source.

The term “reader arrangement” means any detector or detector system capable of detection a signal associated with the binding partner and/or particle, such as an optical signal and/or an electrochemical signal. The reader arrangement may comprise an image acquisition unit e.g. in combination with an optical reader configured for reading an optical signal e.g. of a marker and/or an electrical reader configured for reading an electrochemical signal.

The term “substance” is used to designate any matter that uncountable i.e. not in the form of distinct items. The substance may comprise a homogeneous or inhomogeneous mixture of components and/or elements.

The term “buffer” means an aqueous solution, which is resistant to changes in pH value in the context where the buffer is used. The buffer advantageously comprises an aqueous solution of either a weak acid and its salt or a weak base and its salt.

Unless otherwise specified the pH value of a buffer is determined at 20° C.

The terms “test” and “assay” are used interchangeable.

The term “equilibrium” and “chemical equilibrium” are used interchangeable.

It should be emphasized that the term “comprises/comprising” when used herein is to be interpreted as an open term, i.e. it should be taken to specify the presence of specifically stated feature(s), such as element(s), unit(s), integer(s), step(s) component(s) and combination(s) thereof, but does not preclude the presence or addition of one or more other stated features.

Reference made to “some embodiments” or “an embodiment” means that a particular feature(s), structure(s), or characteristic(s) described in connection with such embodiment(s) is included in at least one embodiment of the subject matter disclosed. Thus, the appearance of the phrases “in some embodiments” or “in an embodiment” in various places throughout the specification is not necessarily referring to the same embodiment(s). Further, the skilled person will understand that particular features, structures, or characteristics may be combined in any suitable manner within the scope of the invention as defined by the claims.

The term “substantially” should herein be taken to mean that ordinary product variances and tolerances are comprised.

Throughout the description or claims, the singular encompasses the plural unless otherwise specified or required by the context.

All features of the invention and embodiments of the invention as described herein, including ranges and preferred ranges, may be combined in various ways within the scope of the invention, unless there are specific reasons not to combine such features.

It has been found that the method and apparatus for determining a characteristic property of a molecular interaction may provide very accurate determinations and in addition, embodiments of the method may be used for performing different and complex determinations, such as determinations of characteristic property or properties of macro particles with a desired high accuracy.

The method of the invention comprises
providing a liquid sample comprising a particle capable of being in a state of equilibrium and in a state of non-equilibrium, the particle comprises a marker in at least one of its state of equilibrium and state of non-equilibrium,
bringing the particle in a state of non-equilibrium by subjecting the sample to a condition jump,
reading out the marker as a function of time during at least a portion of a relaxation time for the particle, and
determining the characteristic property of the molecular interaction,

The step of subjecting the sample to the condition jump may advantageously comprise subjecting the sample to a jump in temperature from at least one first temperature to a second temperature and/or by subjecting the sample to a jump in pressure from a first pressure to a second pressure.

The method of measuring very rapid reaction rates using temperature jump also, referred to as T-jump, is one of a class of chemical relaxation methods pioneered by the German physical chemist Manfred Eigen in the 1950s. In these methods, a reacting system initially at equilibrium is perturbed rapidly and then observed as it relaxes back to equilibrium.

The condition jump and the reading out is advantageously performed in a capillary channel of a microfluid unit as described further below. For example the condition jump may be performed in a first part of the capillary channel (e.g. an introduction section) and the reading out is performed in a second section of the capillary channel (a reading out section).

Generally it is desired that the reading out of the marker as a function of time during at least a portion of a relaxation time for the particle comprises at least two and preferably at least 5, such as at least 8 readings as a function of time from the point of time where the particle is subjected to condition jump, preferably without any intermediate condition jumps.

In an embodiment, the method comprises
providing a liquid sample comprising a particle capable of being in a state of equilibrium and in a state of non-equilibrium, the particle comprises a marker in at least one of its state of equilibrium and state of non-equilibrium,
bringing the particle in a state of non-equilibrium by subjecting the sample to a condition jump comprising a jump in temperature from at least one first temperature to a second temperature,
reading out the marker as a function of time during at least a portion of a relaxation time for the particle, and
determining the characteristic property of the molecular interaction,

wherein the jump in temperature is performed by conduction and/or convection, preferably in a microfluidic unit.

The inventor of the present invention has found that where the jump in temperature is performed by conduction and/or convection a very homogeneous heating may be obtained, which add to increase the accuracy of the determined characteristic property. For example, when heating by subjecting the sample to a pulse of electrical discharge at high voltage and/or optically, the sample may have local hot spots, which may reduce accuracy for some determinations. It was found that in-particular laser heating induces undesired hot-spots, which may deteriorate the measurement and may even damage the sample.

Preferred methods of performing the jump in temperature by conduction and/or convection are described below.

In an embodiment, the method comprises
providing a liquid sample comprising a particle capable of being in a state of equilibrium and in a state of non-equilibrium, the particle comprises a marker in at least one of its state of equilibrium and state of non-equilibrium,
bringing the particle in a state of non-equilibrium by subjecting the sample to a condition jump
reading out the marker as a function of time during at least a portion of a relaxation time for the particle, and
determining the characteristic property of the molecular interaction,

wherein the condition jump comprises subjecting the sample to a jump in temperature from at least one first temperature to a second condition at a second temperature and the method further comprises maintaining the second temperature during at least a part of the reading out of the marker, preferably in a microfluidic unit.

Advantageously the maintaining of the second temperature during at least a part of the reading out of the marker comprises maintaining the temperature within a temperature range of about 2° C., such as within a temperature range of about 1° C., such as within a temperature range of about 0.5° C., such as within a temperature range of about 0.1° C. from the second temperature.

The inventor of the present invention has found that by maintaining the second temperature during at least a part of the time of reading out of the marker, the accuracy of the determined characteristic property may be increased, since otherwise the temperature of the sample may start changing e.g. changing back towards the first temperature, which may provide modified equilibrium conditions and hence, may reduce accuracy. Preferred methods of maintaining the second temperature during at least a part of the time of reading out of the marker are described below.

In an embodiment, the method comprises
providing a liquid sample comprising a particle capable of being in a state of equilibrium and in a state of non-equilibrium, the particle comprises a marker in at least one of its state of equilibrium and state of non-equilibrium,
bringing the particle in a state of non-equilibrium by subjecting the sample to a condition jump comprising a jump in temperature from at least one first temperature to a second temperature and/or by subjecting the sample to a condition jump comprising a jump in pressure from a first pressure to a second pressure,
reading out the marker as a function of time during at least a portion of a relaxation time for the particle, and
determining the characteristic property of the molecular interaction,

wherein the reading out comprises reading out as a function of time comprising performing two or more readings shifted in time and from different fractions of the sample, which has been subjected to the condition jump, preferably in a microfluidic unit.

The inventor of the present invention has found that where the reading out as a function of time comprises performing two or more readings from different fractions of the sample, the risk of degrading the sample and/or the marker of the sample may be reduced. Where readings are performed on the same sample fraction the sample fraction or parts thereof may degrade, thereby resulting in a decrease in accuracy.

This effect of degrading is in particular relevant where the reader arrangement comprises an optical readout. Such optical readout may result in a degradation of the sample, such as of the marker of the sample by photobleaching. By performing two or more readings from different fractions of the sample, the risk of photobleaching may be reduced. Advantageously at least about half of the readings are performed from respective sample fractions that differs from each other.

Advantageously each reading is performed on “fresh” sample fraction that has not previously been read out on.

Preferred methods of performing two or more readings from different fractions of the sample are described below.

In an embodiment, the condition jump comprises a jump in pressure. Using a pressure jump to bringing the particle in a state of non-equilibrium requires a relatively large pressure jump depending on the particle and the molecular interaction in question.

Advantageously the difference between the first and the second pressure is at least about 1 bar, such as at least about 3 bars, such as at least about 10 bars, such as at least about 25 bars.

In practice, a pressure jump below 1 bar will not be sufficient to bringing the particle in a state of non-equilibrium. Suitable pressure jumps are preferably in the range from about 5 bars to about 200 bars, such as from about 20 bars to about 150 bar.

In an embodiment, the particle being capable of being in a state of equilibrium and in a state of non-equilibrium in that the sample comprises a binding partner for the particle or in that, the particle has a structure that depends on temperature and/or pressure. The particle and the binding partner may in practice comprise any interacting molecules, where it is relevant to determine a characteristic property of a molecular interaction between the particle and the binding partner.

The particle may for example comprise a drug or a toxin or a candidate for a drug and the binding partner may for example be a biological compound naturally present in a living being, such as a mammal. In another embodiment, the binding partner may comprise a drug or a toxin or a candidate for a drug and the particle may be a biological compound naturally present in a living being, such as a mammal.

In an embodiment, the particle has a structure that depends on temperature and/or pressure, wherein the particle has a structure at equilibrium at the second condition, which differs from its structure prior to the condition jump.

Advantageously a change of structure of the particle from prior to the condition jump to the structure that the particle will have at equilibrium at the second condition is an at least partly reversible change.

In an embodiment, the particle has a conformation at equilibrium at the second condition, which differs from its conformation prior to the condition jump.

A conformational change is herein used to mean a change in the shape of a molecule, such as a macromolecule, which is induced by the condition jump

A macromolecule is usually flexible and dynamic. It can change its shape in response to changes in its environment or other factors; each possible shape is referred to as a conformation, and a transition between them may be referred to as a conformational change. In an embodiment, the conformational change induced by the condition jump is a structural change, such as a change of a folding where the particle comprises a protein.

In the embodiment of the method where the sample comprises a binding partner for the particle, it may be desirable that at least one of the particle or the binding partner comprises one or more marker. The marker may be any marker capable of being read by the reader arrangement.

Examples of suitable markers are further described below.

The particle or particle and binding partner may or may not be in equilibrium prior to performing the condition jump. Advantageously, the condition jump is sufficient to bring the particle or particle and binding partner to change towards an equilibrium state, which differs from a state at equilibrium at the condition prior to the condition jump.

In a preferred embodiment, the liquid sample comprises the particle and the binding partner in chemical equilibrium or the particle in chemical equilibrium at the time of initiating the condition jump. Thereby the step of bringing the particle in a state of non-equilibrium may be more controlled and the determination of the characteristic property may be more accurate and in addition it may be determined faster than where the particle and the binding partner or the particle is/are not in chemical equilibrium at the time of initiating the condition jump.

Advantageously, the method comprises maintaining the sample at a constant temperature for at least about 30 second prior to performing the temperature jump. Thereby the particle/the particle and the binding partner may be at or be close to equilibrium. Preferably, the method comprises maintaining the sample at a constant temperature for at least about 1 minute, such as at least about 5 minutes, such as at least about 10 minutes prior to performing the temperature jump.

The time for reaching equilibrium may be from seconds to hours, depending of the particle, optional binding partner and the transition, e.g. the conformational change, to reach equilibrium.

The particle may be any kind of particle capable of performing an at least partly chemical or structural transition e.g. a conformational change alone or together with a binding partner.

The liquid sample preferably comprises a liquid buffer system containing the particle or the particle together with the binding partner. The buffer system is advantageously selected to have a pH value, which does not damage or degrade the particle or optional binding partner. The pH value of the buffer system may advantageously be selected in dependence of the molecular interaction to be examined. In an embodiment—in particular where the particle comprises a biopolymer—the pH value is from about 4 to about 9, such as from about 5 to about 8.

In an embodiment, the particle comprises an organic molecule, a cluster of molecules, an aggregate of molecules a nanoparticle, a liposome vesicle, a micelle or any combinations comprising one or more of these.

In an embodiment, the particle comprises a biomolecule; a protein, such as an antibody (monoclonal or polyclonal), a nanobody, an antigen, an enzyme and/or a hormone; a nucleotide; a nucleoside; a nucleic acid, such a RNA, DNA, PNA or any fragments thereof and/or any combinations comprising at least one of these.

A nanobody is an antibody fragment consisting of a single monomeric variable antibody domain. Like a whole antibody, it is able to bind selectively to a specific antigen.

In an embodiment, the molecular interaction comprises liquid-liquid phase interaction, such as liquid-liquid phase separation (LLPS). Liquid-liquid phase separation is a phenomenon that is found in various biological system and which has large importance for biological functions. For example, many membrane-less organelles in living cells and structures are formed by liquid-liquid phase separation.

The list of cell compartments thought to be formed via the process of LLPS is growing rapidly and touches myriad cell functions. In addition to punctate membraneless bodies, other subcellular structures are also formed via LLPS and share similar underlying interactions and physical properties.

Understanding the biophysical principles underlying the formation of biomolecular LLPS is vital for investigation of the physiology and pathophysiology of a wide range of biological processes and systems. Also, for diagnostically purpose and for industrial purpose—e.g. in the food and pharmaceutical industry—there is a need for an improved, rapid and simpler identification and characterization of different biological and non-biological liquid-liquid phase separation systems.

As described and exemplified below the method of embodiments of the invention provides an improved, rapid and simpler method for identification and characterization of liquid-liquid phase separation systems.

Where the molecular interaction comprises a liquid-liquid phase separation, the condition jump is advantageously a temperature jump comprising a jump in temperature from at least one first temperature to a second temperature and wherein the particle comprises at least two different molecules and an optional additional solvent, which molecules are capable of forming a liquid-liquid phase separation at the condition prior to or after the temperature jump.

For example the at least two different molecules may comprise at least one protein, such as an antibody or an enzyme; at least one polymer, such as polyethylene glycol (PEG) or a PEGylated molecule; at least one lipid, such as phospholipid or cholesterol and/or at least one glycosaccharide, such as dextran. In an embodiment, one or more of the two or more different molecules is/are biomolecules. In an embodiment, at least one of the two or more different molecules is a salt in dissociated stage.

The solvent may be an organic solvent, water or an organic solvent-water mixture. Advantageously the organic solvent of the solvent-water mixture is partly or fully miscible with the water at the condition prior to the temperature jump.

Advantageously, the liquid sample immediately prior to subjecting the sample to the temperature jump is in a single phase condition. Thereby it is simple to ensure that the withdrawn and used sample is a representative sample. If the sample is in two or more phases, it may be difficult to withdraw a representative amount of the respective phases from the mother sample to be applied as the sample subjected to the temperature jump.

To ensure that the sample immediately prior to subjecting it to the temperature jump is in a single phase condition, it is desired that the temperature jump is a jump from a higher temperature to a lower temperature. For example the sample may be in a single phase condition at the higher temperature and may be subjected to liquid-liquid phase separation when being subjected to the temperature jump to a lower temperature, e.g. a temperature jump in the temperature interval where the sample is not frozen and not boiling, such as between 90 and 5° C., such as a temperature jump spanning over 5 to 40° C., e.g. 15 to 30° C., e.g. 20-25° C., for example a temperature jump from 50° C. to 25° C.

The induced liquid-liquid phase separation may comprise at least local formation of a first liquid phase with an interface to a second liquid phase.

When performing an assay involving liquid-liquid phase separation, starting at a first higher temperature where the sample is in single phase condition and subjects the sample to a temperature jump to a lower temperature, the first sign of liquid-liquid phase separation may show as sprinkles and/or bobbles of one phase in the remaining portion of the sample. The bobbles may gradually grow as a function of time from the temperature jump e.g. to full separation in phases.

In an embodiment, the sample is in single phase condition is a sample withdrawn from mother sample held stable at the higher temperature. The mother sample may be subjected to stirring or shaking e.g. to maintain the sample a single phase condition.

A marker, such as the marker described elsewhere herein may be bound or inherent in one or more components of the sample. It has been found that upon formation of sprinkles and/or bobbles the signal that may be detected e.g. a fluorescence intensity reflects such formations e.g. by spikes in the signal and/or a change of signal level e.g. intensity. Thereby characteristic properties of liquid-liquid phase separation of various samples a various condition may be determined. This provides a very fast and attractive method of examining formations and stability of liquid-liquid phase separation such as biomolecular LLPS.

The first liquid phase and the seconds liquid phase as well as further liquid phases mays from each other in any way, for example the phases may differ with respect to concentration and/or presence of at least one molecule, such as one of the at least two molecules, such as concentration of dissolved salt. The phases may have same or different solvents, the pH value may differ and/or the phases may differ with respect to hydrophility/hydrophobicity. In an embodiment, the lipid concentration is higher in one phase than in another phase. In an embodiment, the protein concentration is higher in one phase than in another phase.

In an embodiment, the content of the sample is known and the assay has the purpose of determining at least one characteristic of the sample.

In an embodiment, the content of the sample is unknown and the assay has the purpose of determining at least a part of its content, by determining at least one characteristic of the sample and comparing to determined characteristics of known samples.

The characteristic property of the liquid-liquid phase separation may for example comprise one or more of the ability for forming the liquid-liquid phase separation e.g. in dependence of temperature, of concentration of one or more molecules, presence of one or more additional molecule, pH value, concentration of salt in dissociated form.

In an embodiment, where the content of the sample is unknown the method may comprise identifying a fraction of sample capable of forming liquid-liquid phase separation at a selected condition after the temperature jump, the sample may e.g. be an inhomogeneous sample.

The method may further comprise isolating a target portion of the sample from the remaining part of the sample, wherein the target portion of the sample is a portion that has at least one sign of formation of liquid-liquid phase separation. Thereby, where the sample is inhomogeneous, fractions with high ability of forming LLPS may be obtained.

Where the sample is subjected to the temperature jump in the channel of the microfluidic unit and the reading out is performed in the channel, the sample may advantageously be fed to the channel at a pressure ensuring a selected velocity of the sample in the channel. The velocity may conveniently be adjustable, such as adjustable in dependence of the liquid-liquid phase separation status determined by the reading outs.

The method may further comprise acquiring images of at least one local section of the channel. For example, the formation of spikes and/or bobbles may be imaged. It may be desirable to reduce velocity or fully stop the flow at the time of acquiring the image.

The volume of the sample may be relatively small, therefore it may be simpler to prepare a larger volume of mother sample, which may then be used for several examination of the particle in the sample. In an embodiment, the method comprises preparing at least one mother sample and withdrawing the sample from the mother sample.

The volume of the sample is advantageously relatively small. Thereby, it is simpler and faster to perform the condition jump, in particular where the condition jump comprises a temperature jump. In addition, the temperature jump may be a jump to a homogeneous second temperature in the entire sample, which adds to obtain a high accuracy in the determination of the characteristic property.

Advantageously, the sample has a volume of from about 0.1 nl to about 1 ml, such as from about 0.1 μl to about 0.5 ml, such as from about 1 μl to about 0.1 ml.

In an embodiment, the method comprises performing the temperature jump from the at least one first temperature to the second temperature and or the pressure jump from the first pressure to the second pressure in a jump time having a time extend, which is less than the time required for the sample to reach equilibrium at the second condition, preferably jump time is less than two times the time for the sample to reach equilibrium, preferably the jump time is about 1 minute or less, such as about 30 second or less, such as about 10 seconds or less.

In principle it is desired that the time extend for performing the condition jump is as short as possible. The shorter the time extend for performing the condition jump, the longer will the time from the condition jump to equilibrium at the second condition be. Thereby the length of time for performing the readings may be longer and this may add to obtain the desirable high accuracy relatively fast.

A time extend for performing the condition jump of 0.1 to 10 seconds has been found to be very effective.

The condition jump time may be determined from initiating of the temperature jump and/or pressure jump to the time where the entire sample has reached the second temperature and/or the second pressure.

To ensure a relatively long time for performing the reading it has been found desirable to perform the condition jump in the microfluidic unit. Therefore, in an embodiment, the jump in temperature and/or pressure of the sample is performed in the microfluidic unit, the method comprises introducing the sample into the microfluidic unit, wherein the microfluidic unit is preferably at least partly located in a temperature controlled maintaining compartment.

The microfluidic unit may for example comprise an introduction section to which the sample is introduced. The introduction section may advantageously have at least one narrow dimension to ensure that the condition jump of the sample in the introduction section may be performed relatively fast.

The introduction section may advantageously comprise a cross-sectional dimension of about 1 mm or less, such as of about 0.5 mm or less, such as of about 0.1 mm or less, such as of about 75 μm or less.

In an embodiment, the introduction section comprises a flat chamber, a channel, two or more interconnected channels or any combinations comprising one or more of these.

A flat chamber is advantageously a chamber having a height dimension, which is 50% or less than at least one of its width and length.

The introduction section has a volume, which is preferably at least as large as the sample. In addition, it is desired that the introduction section is not too much larger than the sample. Advantageously it has a volume corresponding to the volume of the sample or up to about 20% larger.

The volume of the introduction section of the microfluidic unit may for example be from about 0.1 nl to about 1 ml, such as from about 0.1 μl to about 0.5 ml, such as from about 1 μl to about 0.1 ml.

In an embodiment, the volume of the introduction section is defining the volume of the sample and/or the introduction section is defined by the volume of the sample. I.e. the volume of the microfluidic unit filled by the sample at the time of performing the condition jump is defined to be the introduction section of the microfluidic unit.

Advantageously, the temperature controlled maintaining compartment is maintained at the second temperature and/or at the second pressure during at least a portion of the relaxation time, preferably during at least a part of the reading out, to thereby ensure a stable second condition.

The temperature controlled maintaining compartment may for example be temperature controlled by a method comprising blowing of air, preferably air having the second temperature. It should be understood that any other gas than air may be used instead of or in combination with air.

In an embodiment, the temperature controlled maintaining compartment is temperature controlled by a method comprising fully or partly filling the compartment with liquid and/or vapor, preferably having the second temperature.

In an embodiment, the temperature jump is performed by a method comprising blowing air, or flowing liquid over a container containing the sample, e.g. the where the container form part of or comprises at least a part of the microfluidic unit as explained above.

In an embodiment, the temperature jump may be performed by a method comprising applying a high voltage to the sample (e.g. using a pulse and/or Joule heating), preferably while the sample is located in a container, such as a container, which form part of or comprises at least a part of the microfluidic unit, such as while the sample is located in the introduction section of the microfluidic unit.

The high voltage may be applied as a pulse of electrical discharge at the high voltage. As explained above using a pulse of electrical discharge at high voltage, may result in the formation of local hot spot in the sample. However, for some molecular interactions, the time from performing to temperature jump to equilibrium is relatively long, and by ensuring that the sample volume is relatively small, the heat alt the local hot spot may be dissipated to the entire sample relatively fast, thereby ensuring that a determination at an acceptable and even relatively high accuracy may be performed.

In an embodiment, the temperature jump is performed by a method comprising applying a joule heating element (e.g. applying a substantially continues high voltage through the sample for at least 0.1 second and until the desired temperature is reached), a resistive element and/or a peltier element to conduct heat to the sample. The conduction of heat to the sample is advantageously performed while the sample is located in a container, such as a container, which form part of or comprises at least a part of the microfluidic unit, such as while the sample is located in the introduction section of the microfluidic unit. Preferably, the joule heating element, resistive element and/or peltier element is located in physical contact with the container.

Joule heating elements, resistive elements and peltier elements are known to the skilled person and the skilled person will be able to select a suitable joule heating element, resistive element and/or peltier element based on the teaching presented herein.

In an embodiment, the pressure jump is performed by a method comprising locating the sample in a container comprising a membrane, such as a polyimide membrane (e.g. a kapton membrane), wherein a piezoelectric crystal stack is arranged to depress the membrane, wherein the pressure jump is performed by activating the piezoelectric crystal stack to increase the pressure or to deactivate the piezoelectric crystal stack to decrease the pressure. The container used as microfluidic unit where the condition jump is performed as a pressure jump is advantageously of a strong material such as sapphire e.g. synthetic sapphire (crystallized aluminum oxide). The sample may be injected to flow into the microfluidic unit via the membrane and optical read out may e.g. be performed via the sapphire.

In an embodiment, the temperature jump is performed by a method comprising mixing the sample with additional liquid at a selected temperature different from the first temperature. This method may be performed in a T-shaped flow cell as the microfluidic device, such as the microscale channel cells described in U.S. Pat. No. 5,972,710.

In an embodiment, the additional liquid is preferably free of the particle and the binding partner. Thereby the sample becomes a diluted sample.

In an embodiment, the method comprises providing the sample in the form of two or more sub-samples having different first temperatures and wherein the temperature jump is performed by a method comprising bringing the two or more sub-samples together, for example in adjacent laminar flow or by mixing. The two or more sub-samples may have equal or different concentration(s) of particle and/or binding partner.

In an embodiment, the relative concentration of particle and binding partner in each of the sub-samples are identical, preferably the concentration of particle and binding partner in each of the sub-samples are essentially identical, more preferably the chemical composition of the sub-samples are identical.

The temperature jump from at least one first temperature to the second temperature advantageously comprises providing a temperature jump of at least about 2° C., such as at least about 5° C., such as at least about 10° C., such as at least about 15° C.

The minimum temperature jump for bringing the particle in a state of non-equilibrium depends on the molecular interaction examined and the concentration of the particle and optional binding partner.

For many molecular interactions, a temperature jump of from about 5° C. to about 30° C. may be suitable. For LLPS assays, a temperature jump from high to low temperature, such as from 40-50° C. to about 20-25° C. may be advantageous.

For the molecular interaction examination, the second temperature may be important for the characteristic property to be determined. If for example the characteristic property correlates to a property of the particle in a specific temperature range—e.g. a property of a drug within a living being—the second temperature is advantageously selected to be within that specific temperature range.

The second temperature may be higher or lower than the at least one first temperature. In many situations, it may be simpler to perform the temperature jump from a lower to a higher temperature, e.g. where the temperature jump is performed using a heating element.

The second temperature may advantageously be from about 5° C. to about 50° C., such as from about 10° C. to about 45° C., such as from about 20° C. to about 42° C., such as from about 35° C. to about 40° C., e.g. from 25-37° C.

In practice a second temperature at or within 5° C. from a natural temperature of a living being may be desirable.

In an embodiment, the method comprises introducing the sample into the microfluidic unit at a pressure difference of at least about 0.1 bar, such as at least about 0.2 bar, such as at least about 0.3 bar as at least about 0.4 bar as at least about 0.5 bar, such as at a pressure difference less than 1 bar, such as less than 0.9 bar.

In an embodiment, the method comprises introducing the sample into the microfluidic unit at a pressure of from about 0.5 to about 3 barg,

The sample is advantageously introduced in the microfluidic unit, e.g. an introduction section of the microfluidic unit relatively fast, where it is subjected to the condition jump, such as the temperature jump. The microfluidic unit may be preheated, such that the temperature jump is initiated immediately as the sample in introduced into the microfluidic unit.

The microfluidic unit may in principle have any shape but is advantageously shaped as described herein. In an embodiment, the microfluidic unit comprises a flat chamber, a channel, two or more interconnected channels or any combinations comprising one or more of these.

In an embodiment, the microfluidic unit comprises a channel and preferably is in the form of a tube or a chip, wherein the channel preferably has a cross-sectional dimension of about 1 mm or less, such as of about 0.5 mm or less, such as of about 0.1 mm or less, such as of about 75 μm or less, preferably the channel has a maximal cross-sectional dimension of about 1 mm or less, such as of about 0.5 mm or less, such as of about 0.1 mm or less, such as of about 75 μm or less. The microfluidic unit may for example be shaped as a tube with equal diameter in its entire length. Such tube is also referred to as a capillary tube.

In an embodiment, the microfluidic unit comprises an introduction section e.g. ad described above and a reading out section. The introduction section and the reading out section may be directly in length connection of each other.

In an embodiment, the introduction section and the reading out section are at least partially overlapping. The reading out may be performed while the sample is located in the same location where it had been subjected to the condition jump.

In an embodiment—which is preferred, the introduction section and the reading out section are distinct sections.

In an advantageous embodiment, the method comprises flowing at least a part of the sample from the introduction section to the reading section.

In an embodiment, the reading out comprises performing readings of the sample while the sample is stationary (non-flow condition) in the microfluidic unit. As described above the readings are preferably performed from different fractions of the sample. This may for example be performed by moving the reader arrangement and the microfluidic unit relative to each other.

In a preferred embodiment, the reading out comprises performing readings of the sample while the sample is flowing in the microfluidic unit. Preferably, the reading out as a function of time comprises performing the two or more readings from different fractions of the sample as the sample is flowing in the reading section of the microfluidic unit. Thereby the reader arrangement may perform the readings from different fractions of the sample without this requires mowing the reader arrangement and the microfluidic unit relative to each other. Usually moving elements in an apparatus may add to the complexity and cost of the apparatus. Hence, the method comprising performing readings of the sample while the sample is flowing in the microfluidic unit provides to improve the cost effectivity of the method and the apparatus for performing the method.

The flow velocity of the sample in the reading out section may advantageously be adjusted to the reading rate, so that the desired number of reading may be performed.

Advantageously, the method comprises adjusting the flow velocity at location(s) of reading out to be up to about 50 cm/sec, such as up to about 25 cm/sec, such as up to about 10 cm/sec, such as up to about 2 cm/sec, such as up to about 1 cm/sec, such as up to about 0.1 cm/sec.

The reading rate may e.g. be at least about 5 readings per minute, such as at least about 10 readings per minute, such as at least about 30 readings per minutes, such as at least about 60 readings per minutes, such as at least about 120 readings per minute.

A reading rate of from about 1 reading to 30 readings per second may be suitable for most determinations.

Advantageously the reading out as a function of time comprises performing consecutive readings from different fractions of the sample as the respective sample fractions are passing a reading location of the microfluidic unit.

The method may advantageously comprise introducing the sample into the microfluidic unit at a first higher pressure, such as at a pressure difference up to 1 bar e.g. as described above. After or during the introduction the condition jump may be performed. If the condition jump is performed after the sample is fully introduced, the pressure difference may be reduced or terminated, such that the sample in non-flowing during the condition jump.

This embodiment is advantageous when the condition jump comprises a temperature jump.

If the condition jump comprises a temperature jump it is advantageous that the temperature jump is performed during the introduction of the sample into the introduction section. The microfluidic unit may advantageously be preheated. After the condition jump, the method advantageously comprises reducing the pressure to a second lower pressure.

The second lower pressure may be as described above. For example the second lower pressure advantageously is at least about 10% lower than the first higher pressure, such as at least about 25% lower than the first higher pressure, such as at least about 50% lower than the first higher pressure, such as at least about 75% lower than the first higher pressure, such as at least about 90% lower than the first higher pressure, such as at least about 95% lower than the first higher pressure, such as at least about 99% lower than the first higher pressure.

The marker may be any marker capable of being read by the reader arrangement e.g. as described above. The marker may be an intrinsic marker, an extrinsic marker or a combination thereof.

Where the particle comprises a biomolecule, it is often desired to use an intrinsic marker, such as intrinsic tryptophan fluorescence or absorbance.

Advantageously, the marker is sensitive to the molecular interaction, such a sensitive to a conformational change of the particle, preferably the marker changes signal in dependence of conformation of the particle and conformational changes thereof, such as in dependence of a change in binding/dissociation and/or a change in structure.

In an embodiment, the marker is sensitive to protein interactions—for example, the signal changes upon binding/dissociation.

In an embodiment, the marker is an optically readable marker, such as a light absorbing marker and/or a fluorescent marker, preferably operating in the UV/Vis wavelength range preferably from about 190 nm to about 700 nm.

The marker may for example comprises a quencher.

In particular where the marker needs excitation, there may be a risk high risk of photobleaching if a plurality of readings is performed on the same sample fraction. Hence, it may be preferred to ensure that the method comprises performing two or more readings from different fractions of said sample as described elsewhere herein.

In an embodiment, the marker is an electrochemically readable marker, such as an electroactive marker. A non-limiting example of an electrochemically readable marker is an osmium tetroxide marker.

The reading out of the marker as a function of time during at least a portion of a relaxation time advantageously comprises performing a plurality of consecutive readings of the marker. The readings preferably comprise reading(s) of electrode potential, reading(s) of intensity of one or more wavelengths and/or reading(s) of change of one or more wavelength(s).

The change of one or more wavelength(s) may for example be a wavelength shift.

In an embodiment, Fluorescence Resonance Energy Transfer (FRET) and/or Bioluminescence Resonance Energy Transfer (BRET) are used to monitor the distances between two markers, where one marker is on or is associated to the particle and another of the markers is on or is associated to the binding partner.

The plurality of readings advantageously comprises at least 5 readings, such as at least 10 readings, such as at least 50 readings, such as at least 50 readings or more.

Advantageously, the method comprises performing a plurality of consecutive readings of the marker until the consecutive readings changes less than about 25% from one reading to the next, such as until the consecutive readings changes less than about 10%, such as until the consecutive readings changes less than about 5%, such as until the consecutive readings changes less than about 1%, preferably until relaxation is reached. It may not be required to continue the readings until full relaxation, however, in practice it may be simpler and/or safer to continue readings until full relaxation.

In an embodiment, the method further comprises performing the method one or more additional times using different temperature jump and or using different concentration(s) of the particle and or the binding partner and preferably determining additional characteristic property of the molecular interaction.

The method may be applied for determine any conformational change such as protein foldings and or any kinetic reactions between a particle and a binding partner.

In an embodiment, the method comprises determining at least one of a kinetic parameter, such as Kd; a partitioning parameter, such as formation/deformation of liposome or micelle; a degradation parameter; an oligomerization parameter; a folding parameter, such as unfolding or refolding, a multi-binding parameter, such as a parameter representing multiple binding by distinct timescales.

In an embodiment, the method comprises determining a characteristic property of molecular interaction(s) between a particle and two or more binding partners and/or two or more particles and a binding partner

The characteristic property of the molecular interaction may for example comprises determining at least one kinetic parameter, such as equilibrium constant (Kd value) of the at least one particle and/or the at least one particle and the at least one binding partner, such as determining an affinity between the at least one particle and the at least one binding partner and/or determining of one of both of the kinetic rate constants kon/koff.

Examples of characteristic properties that may be determined includes any kinetic parameters, such as Kd, kon and koff; partitioning, such as in and out of liposome or micelle, LLPS systems, degradation: degradation; oligomerization; unfolding; refolding; multiple binding by distinct timescales and/or particle concentration.

The method as described herein may be combined by other assays such as one or more diffusion assays of the particle or particle and its binding partner. The diffusion assay may for example be applied to determine a particle/binding partner concentration balance, which may be desirable for use in the method, described herein, e.g., where a condition jump may have large effect on the equilibrium/non-equilibrium status of the particle and binding partner.

The diffusion assay may for example be applied to determine a hydrodynamic radius of the particle.

In an embodiment, the diffusion assay is performed at different concentration(s) of at least one of the particle and or the binding partner to determine a concentration wherein at least one of the kinetic rate constants kon/koff is sensitive to a change.

The invention also comprises an apparatus suitable for determining a characteristic property of molecular interaction.

The apparatus comprises
a sample compartment for containing at least one liquid mother sample;
a withdrawing arrangement arranged for withdrawing a sample from a at least one mother sample stored in the sample compartment
a condition jump arrangement, and
at least one reader arrangement for reading at least one marker as a function of time.

The condition jump arrangement is advantageous arranged for performing the condition jump as described above.

In an embodiment, the apparatus comprises
a sample compartment for containing at least one liquid mother sample;
a withdrawing arrangement arranged for withdrawing a sample from a at least one mother sample stored in the sample compartment
a condition jump arrangement arranged for performing a temperature jump of the sample from at least one first temperature to a second temperature, and
at least one reader arrangement for reading at least one marker as a function of time,

wherein the apparatus is adapted for performing the temperature jump by conduction and/or convection, preferably with the sample contained in a microfluidic unit.

Providing that the apparatus is adapted to perform the temperature jump by conduction and/or convection ensures that a very homogeneous heating of a sample may be obtained as it is explained above.

In an embodiment, the apparatus comprises
a sample compartment for containing at least one liquid mother sample;
a withdrawing arrangement arranged for withdrawing a sample from a at least one mother sample stored in the sample compartment
a condition jump arrangement arranged for performing a temperature jump of the sample from at least one first temperature to a second temperature, and
at least one reader arrangement for reading at least one marker as a function of time,
wherein the apparatus further comprises a maintaining compartment for maintaining the sample at the second condition during the reading out of the marker, preferably with the sample contained in a microfluidic unit.

The apparatus may advantageously be adapted for maintaining the temperature within a temperature range of about 2° C., such as within a temperature range of about 1° C., such as within a temperature range of about 0.5° C., such as within a temperature range of about 0.1° C. from the second temperature.

Providing that the apparatus is adapted to maintaining the second temperature during at least a part of the reading out of the ensures the accuracy of the determined characteristic property may be increased as it is explained above.

In an embodiment, the apparatus comprises
a sample compartment for containing at least one liquid mother sample;
a withdrawing arrangement arranged for withdrawing a sample from a at least one mother sample stored in the sample compartment
a condition jump arrangement arranged for performing a temperature jump of the sample from at least one first temperature to a second temperature and/or arranged for performing a jump in pressure from a first pressure to a second pressure, and
at least one reader arrangement for reading at least one marker as a function of time,
wherein the apparatus is adapted for performing the reading out as a function of time by performing two or more readings from different fractions of the sample, preferably with the sample contained in a microfluidic unit.

Providing that the apparatus is adapted to perform the reading out as a function of time by performing two or more readings from different fractions of the sample ensures that the risk of degrading the sample and/or the marker of the sample may be reduced as it is explained above.

The apparatus may advantageously be adapted to perform the method as claimed and as described above.

Advantageously, the sample compartment comprises at least one temperature control arrangement for selecting and controlling the temperature of at least one mother sample located in a mother sample chamber of the sample compartment. The sample compartment may be adapted for or comprises two or more mother sample chambers, wherein the apparatus is adapted for selecting and controlling the temperature of respective mother samples located in the respective mother sample chambers individually or collectively. Thereby the apparatus may be applied, e.g. programmed to perform assays of several equal or different samples one after the other without it requires refilling or changing the mother sample(s).

In an embodiment, the withdrawing arrangement comprises a tool for withdrawing and transporting the sample from the sample to an inlet of the microfluidic unit, such as a manually handled tool.

The tool may for example include a pipette and a user may withdraw the sample (e.g. a drop) and manually move it to an inlet of the microfluidic unit.

This embodiment may be advantageous for users where only few determinations are to be performed, since this may reduce the cost of the apparatus.

Advantageously, the withdrawing arrangement form part of or is in fluid communication with the microfluidic unit.

The withdrawing arrangement may advantageously comprise a pump arrangement adapted for moving (flowing) the sample from the sample compartment to the microfluidic unit. The pump arrangement may be any arrangement capable of transporting the sample from the sample compartment to the microfluidic unit. Preferably, the pump arrangement comprises an electrokinetic driven pump arrangement and/or a pressure-driven pump arrangement, such as a suction pump arranged for sucking the sample into the microfluidic unit and/or a pressure pump arranged for pumping the sample into the microfluidic unit.

Examples of electrokinetic driven pump arrangements may for example be found in Devasenathipathy S, Santiago JG (2004) “Electrokinetic flow diagnostics” Springer, New York Berlin Heidelberg.

The withdrawing arrangement may comprise a tube for withdrawing the sample from the sample compartment. The tube may be multi-furcated to have several tube inlet, which may be arranged to withdraw from respective mother sample chamber. In an embodiment, the tube end or tube ends are adapted for being moved from mother sample container to mother sample container between sample withdrawing respective samples.

The phenomenon of electrokinetics driven flow comprises electroosmosis electrophoresis and streaming potential.

The withdrawing arrangement may be adapted for withdrawing the sample from one single mother sample chamber.

In an embodiment, the withdrawing arrangement is adapted for withdrawing the sample from two or more mother sample chambers.

The withdrawing arrangement may advantageously be configured for feeding the sample to the inlet of the microfluidic unit at a feeding pressure, wherein the feeding pressure is adjustable, such as manually adjustable or controllable by the computer system. The computer system may be programmed to control the velocity of the sample in dependence of time from the condition jump and/or in dependence of the read out signal, preferably in real time.

The computer system may be programmed to control the velocity a function of the read out signal in real time. The phrase “real time” is herein used to mean with less than 1 second delay. For example, the computer may be programmed to slow down velocity for image acquisition and/or for improving reading accuracy where changes in signal exceeds a preset threshold.

The apparatus may comprise an image acquisition unit located for acquiring images of at least a portion of the sample located downstream to a location where it is subjected to the condition jump. The image acquisition unit may be located for acquiring images of at least one local section of the channel, such as a local section located downstream to the reading out location.

The condition jump arrangement may be at least partly integrated with the microfluidic unit. For example, the microfluidic unit may comprise two or more inlets adapted for bringing sub-samples withdrawn from the respective mother sample chambers into contact, e.g. by arranging the sub-samples in layered (e.g. laminar) flow or by mixing the sub-samples as further described above.

Advantageously the condition jump arrangement comprises a heating and/or cooling arrangement adapted for performing the temperature jump from the first temperature to the second temperature.

In an embodiment, the condition jump arrangement comprises a pressure increasing or reducing arrangement adapted for performing the pressure jump from the first pressure to the second pressure.

The apparatus is advantageously adapted to perform the condition jump relatively fast, e.g. with a jump time as described above.

Advantageously, the condition jump arrangement is arranged for performing the jump in temperature and/or pressure of the sample in the microfluidic unit. The condition jump arrangement is preferably at least partly located in the temperature controlled maintaining compartment.

The condition jump arrangement and/or the maintaining compartment preferably comprise a temperature controller arrangement. The temperature controller arrangement may for example comprise a blower for blowing air at a selected temperature and/or a liquid sprinkler for sprinkling liquid at a selected temperature and/or a liquid filler for fully or partly filling the maintaining compartment with liquid at a selected temperature.

In an embodiment, the condition jump arrangement comprises a joule heating arrangement arranged for applying a high voltage to the sample, preferably while the sample is located in a container, such as a container, which forms part of or comprises at least a part of the microfluidic unit, such as while the sample is located in the microfluidic unit, for example in an introduction section of the microfluidic unit.

In an embodiment, the condition jump arrangement comprises a joule heating element, a resistive element and/or a peltier element arranged to conduct heat to the sample, preferably while the sample is located in a container, such as a container, which forms part of or comprises at least a part of the microfluidic unit, such as while the sample is located in the microfluidic unit. Preferably, the joule heating element, resistive element and/or peltier element is located in physical contact with the container.

The reader arrangement may be as described above.

In an embodiment, the reader arrangement may be any kind of reader, which does not performing undesired change of the interaction under analysis.

The at least one reader arrangement comprises an optical reader arrangement and/or an electrochemical reading arrangement.

Advantageously, the at least one reader arrangement is adapted for performing a plurality of readings as a function of time, preferably with a reading rate of at least about 5 readings per minute, such as at least about 10 readings per minute, such as at least about 30 readings per minutes, such as at least about 60 readings per minutes, such as at least about 120 readings per minute.

Advantageously, the at least one reader arrangement is stationary located in the apparatus, the reader arrangement is advantageously adapted for performing readings of markers of sample fractions as the sample fractions passes the reader arrangement, preferably by flowing in the microfluidic unit.

Providing that the reader arrangement is stationary located, may reduce cost of the apparatus e.g. as described above.

The apparatus may advantageously be adapted for controlling the flow rate

The reader arrangement is preferably located for reading out from the microfluidic unit in the maintaining compartment, preferably, at least a reading head of the reader arrangement is located in the maintaining compartment.

The invention also comprises an assembly comprising the apparatus as claimed and as described herein in combination with the microfluidic unit. The microfluidic unit is preferably is at least partly located in the temperature controlled maintaining compartment.

The microfluidic unit may advantageously be as described herein and e.g. comprising a flat chamber, a channel, two or more interconnected channels or any combinations comprising one or more of these.

In an embodiment, the microfluidic unit is adapted to be closed and comprises a membrane wall section and an arrangement for moving the membrane, e.g. using a piezoelectric crystal stack to change the pressure within the microfluidic unit.

The microfluidic unit advantageously comprises a channel. The channel preferably has a length of at least about 1 cm, such as of at least about 10 cm, such as of at least about 25 cm, such as of at least about 50 cm, such as of at least about 75 cm, such as of at least about 1 m or longer. In principle, the channel may be as long as desired, but for most determinations, a channel of from 1 cm to 2 m in length may be sufficient. The channel may be meander folded, coiled or bend in any other desired configurations.

In an embodiment, the microfluidic unit comprises an introduction section and a reading out section. The introduction section and the reading out section may be at least partially overlapping or the introduction section and the reading out section may be distinct sections.

Advantageously, reader arrangement is located to read out from a stationary reading location of the microfluidic unit.

In an embodiment, the apparatus comprising a pump arrangement, e.g. as the pump arrangement described above.

The pump arrangement may for example be adapted for introducing the sample into the microfluidic unit at a first higher pressure difference and reducing the pressure difference to a second lower pressure difference. The pump arrangement may preferably be adapted for maintaining the second lower pressure difference during at least a part of the reading out. The pump arrangement may advantageously comprise a pressure pump and/or a suction pump.

The invention also comprises a system suitable for determining a characteristic property of molecular interaction. The system comprises an apparatus as claimed and/or as described herein or an assembly as claimed and/or as described herein and a computer system. The computer system is configured for
controlling the withdrawing arrangement
controlling the temperature jump and spreading arrangement
controlling the reader arrangement and/or
determining the characteristic property of the molecular interaction.

The system may advantageously be suitable for determining a characteristic property of molecular interaction where the molecular interaction comprises a change of structure of a particle and/or a change in binding between a particle and a binding partner for the particle, preferably where the molecular interaction comprises a change of conformation.

In an embodiment, the computer system is configured for determining at least one of a kinetic parameter, such as Kd; a partitioning parameter, such as formation/deformation of liposome, formation/deformation of micelle and/or liquid-liquid phase separation or unification; a degradation parameter;

an oligomerization parameter; a folding parameter, such as unfolding or refolding, a multi-binding parameter, such as a parameter representing multiple binding by distinct timescales.

In an embodiment, the computer system is configured for determining a characteristic property of molecular interaction(s) between a particle and two or more binding partners and/or two or more particles and a binding partner.

In an embodiment, the computer system is configured for determining at least one kinetic parameter, such as equilibrium constant (Kd value) of the at least one particle and/or the at least one particle and the at least one binding partner, such as determining an affinity between the at least one particle and the at least one binding partner and/or determining of one of both of the kinetic rate constants kon/koff.

In an embodiment, the computer system is configured for controlling the performance of the method according to any one of claims 1-60.

All features of the invention(s) and embodiments thereof including ranges and preferred ranges can be combined in various ways within the scope of the invention, unless there are specific reasons not to combine such features.

### BRIEF DESCRIPTION OF THE EXAMPLES AND DRAWING

The invention is being illustrated further below in connection with examples and embodiments and with reference to the figures. The figures are schematic and may not be drawn to scale. The examples and embodiments are merely given to illustrate the invention and should not be interpreted to limit the scope of the invention

FIG. 1 illustrates an embodiment of a system of the invention comprising a computer system and an assembly of an apparatus and a microfluidic unit.

FIG. 2 illustrates a variation of the embodiment in FIG. 1.

FIGS. 3a-3e show examples of microfluidic units suitable for use in embodiments of the apparatus of the invention.

FIGS. 4a and 4b are diagrams showing a fluorescence intensity as a function of time as described in example 1.

FIGS. 5a-5g are diagrams showing a fluorescence intensity as a function of time as described in examples 2a-2g.

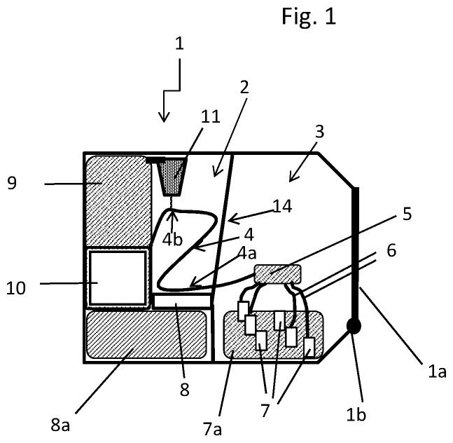

The system of FIG. 1 comprises an apparatus 1 suitable for determining a characteristic property of a molecular interaction and a microfluidic unit 4. The apparatus comprises a maintaining compartment 2 and a sample compartment 3 separated by a separating wall 14 having a passage for the microfluidic unit 4.

The sample compartment 3 comprises a plurality of mother sample chambers 7, arranged in a support unit 7a. The support unit 7a advantageously comprises a temperature controller for temperature controlling of mother samples in the respective mother sample chambers 7 to a selectable temperature. The sample compartment 3 comprises a withdrawing arrangement comprising a pump arrangement 5, connected to a plurality of withdrawing tubes 6. Each tube advantageously comprises a needle adapted for penetrating a cover membrane on the respective of mother sample chambers 7. The respective tubes 6 may be manually inserted into desired mother sample chambers, by penetrating the membrane of the mother sample chamber with the needles at their ends. In an embodiment, the apparatus 1 comprises a robot arm adapted for insert the tube(s) 6 into selected mother sample chamber(s).

In a variation of this embodiment the withdrawing arrangement comprising a single withdrawing tube.

The apparatus 1 comprises a hinged 1b lid 1a into the sample compartment 3 for providing access there to.

In this embodiment, the microfluidic unit 4 is a tube with a narrow diameter e.g. as described above. The tube 4 is connected to the pump arrangement, such that the pump can pump withdrawn mother sample into the microfluidic unit 4 at a desired pressure difference.

The maintaining compartment 2 comprises a computer unit 9 adapted for controlling the elements of the apparatus 1. The computer 9 is connected to a reader arrangement 11.

The maintaining compartment 2 comprises a condition jump arrangement 8, adapted for performing the temperature jump by conduction and/or convection e.g. as described above. The condition jump arrangement 8 may for example comprise a blower or a peltier element. A temperature controller arrangement 8a is connected with the condition jump arrangement 8, such that the temperature controller arrangement 8a may control the operation of the condition jump arrangement 8 and the temperature in the maintaining compartment 2.

A waist chamber 10 is located for collect used sample and optional cleaning fluid passed through the microfluidic unit 4

The microfluidic unit 4 has an introduction section 4a which is arranged adjacent to the condition jump arrangement 8. The microfluidic unit 4 also has a reading out section 4b, which is this embodiment is a single location at the microfluidic unit.

In use, the sample is withdrawn from one or more selected mother sample containers 7 by the tube(s) 6 and the pump arrangement 5 of the withdrawing arrangement.

The sample is fed into the microfluidic unit 4 into the introduction section 4a at a relatively high pressure difference to ensure that the introduction of sample is performed relatively fast. When the sample has reach the introduction section 4a, the pump arrangement, the pressure provided by the pump arrangement 5 is reduced or fully stopped. In the introduction section 4a the condition jump arrangement 8 is heating the sample very fast to ensure a desired temperature jump.

Thereafter, pump arrangement 5 is pumping the sample to reach the read out section 4b. The pressure is reduced to provide that the sample is passing the read out section 4b at a desired slow velocity to ensure a desired long reading timed. While the sample is passing the read out section 4b, the reader arrangement 11 is performing a plurality readings at a desired reading rate e.g. as described above.

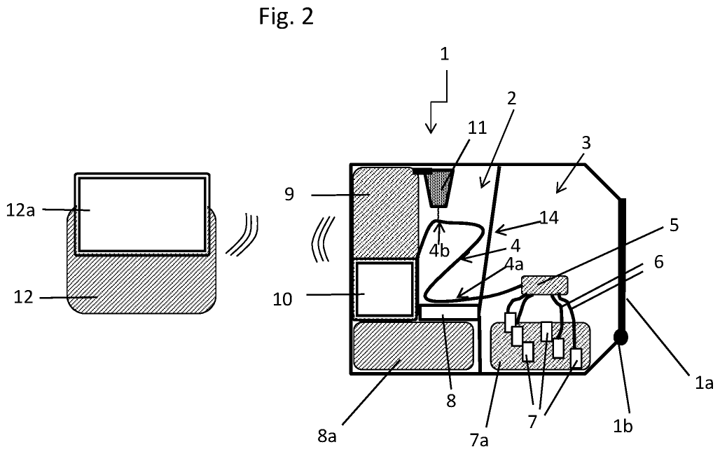

The variation of the system shown in FIG. 2 comprises a personal computer 12, with a screen 12a. The personal computer 12 is in data connection with the computer 9, incorporated in the apparatus 1. The computer system comprises the personal computer 12 and the computer 9.

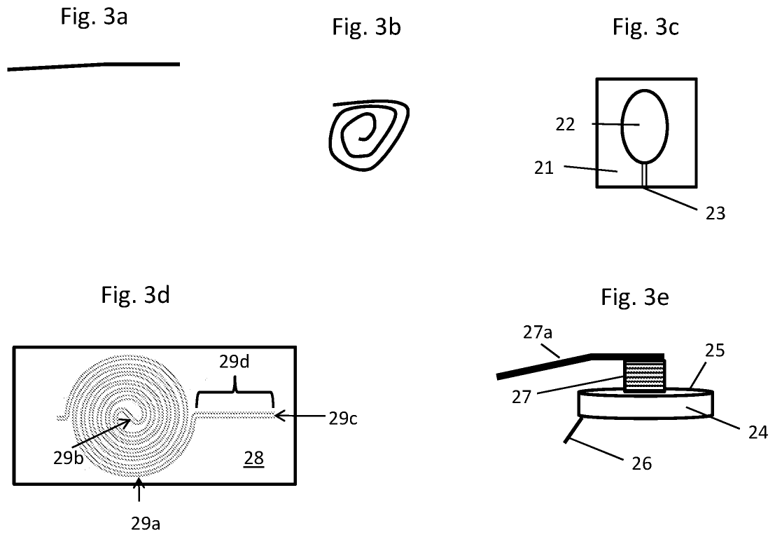

FIG. 3a shows an embodiment of a suitable microfluidic unit in the form a long, substantially straight tube with a narrow inner diameter.

FIG. 3b shows an embodiment of a suitable microfluidic unit in the form a long, coiled tube with a narrow inner diameter.

FIG. 3c shows an embodiment of a suitable microfluidic unit in the form a microfluidic device 21, with a flat chamber 22 and an inlet 23 to the chamber 22.

FIG. 3d shows an embodiment of a suitable microfluidic unit in the form a microfluidic device 28, with a long coiled channel 29a. The channel has an inlet 29c, leading to an introduction section 29d, where a sample may be subjected to a temperature jump. The channel has a reading out section 29b.

FIG. 3e shows an embodiment of a suitable microfluidic unit in the form a chamber provided by crystallized aluminum oxide 24 with a membrane cover 25 and bottom. The sample may be introduced into the chamber via a tube 26. The figure also illustrates a part of the condition jump arrangement adapted for performing a pressure jump. The condition jump arrangement comprises a piezoelectric crystal stack 27 and a holding arm 27a adapted to hold the piezoelectric crystal stack 27 against the membrane 25.

### Example 1—HSA-Fluorescein Binding Partner Assay

A sample comprising a molar concentration of human serum albumin (HSA) of 83 micro mol and a molar concentration of 10 nano mol of a binding partner to the HSA, namely Flourescein (fl) in a buffered solution at a pH value of 7.4.

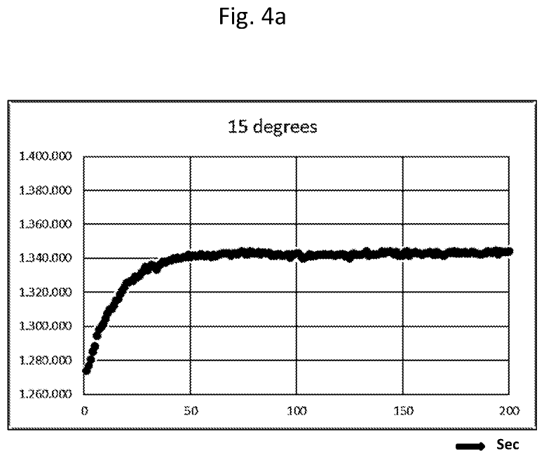

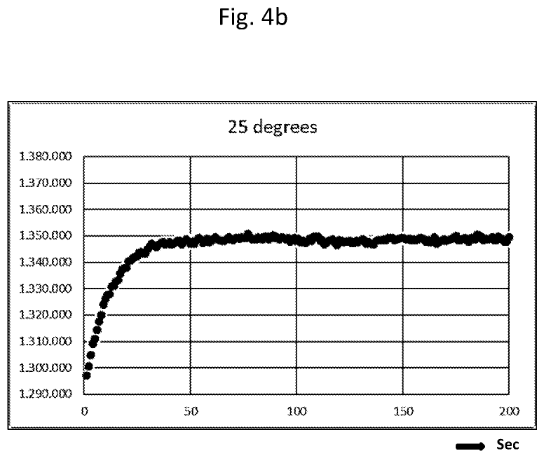

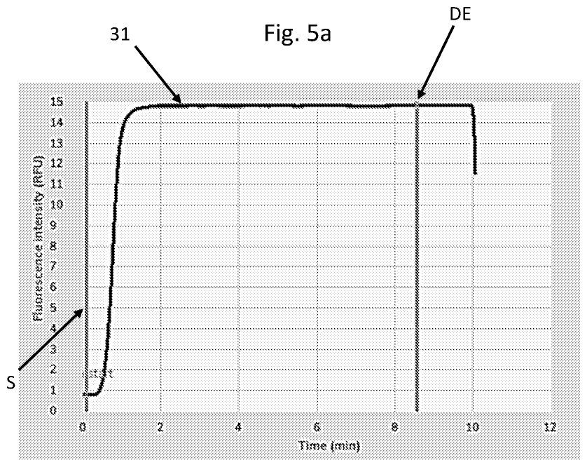

An assay was performed as describe in connection with FIG. 1, where the temperature jump was a 10 degrees jump from 5° C. to 15° C. The resulting readings were plotted and are shown in FIG. 4a.

Another assay was performed as describe in connection with FIG. 1, where the temperature jump was a 20 degrees jump from 5° C. to 25° C. The resulting readings were plotted and is shown in FIG. 4b.

In FIG. 4a, the final temperature is 15° C. and the relaxation to equilibrium is governed by the rate constants at 15° C. In FIG. 4b, the final temperature is 25° C. and the relaxation to equilibrium is governed by the rate constants at 25° C. Kinetic rate constants are higher at higher temperatures compared to lower temperatures. Relaxation kinetics can be described by the relaxation time denoted by tau:

S=a+b(1−exp(−t/tau))

S is the signal obtained from the reader (in this case a fluorescence reader), a is a constant describing detection offset and or background, b is the magnitude of the change in signal between initial state and final state and it is time.

Tau is quantified by and appropriate fit to the data. In a more advanced data analysis, the relaxation may be modeled using several tau values is several relaxation processes are in play.

Tau is linked to the rate constants pertaining to the molecular property under study. For example, a 1-1 non-covalent interaction (A+I=AI) in which A is in large excess of I may be linked to tau according to:

Tau=1/(kon[A]+koff)

In which kon and koff are the rate constants pertaining to formation and dissociation of the complex AI.

### Example 2a—LLPS Assay

A mother sample (a) was prepared.

The following materials were used in this or in the following examples:

FI-dextran: A fluorescently labeled dextran having a molar weight of about 7000 Dalton.

Dextran: A non-labeled dextran having a molar weight of about 200000 Dalton.

PEG: Poly(ethylene glycol), molar weight of about 6000 Dalton.

Water: Pure water (type II).

FI-HSA: A fluorescently labeled Human Serum Albumin.

An aqueous mother sample (a) were prepared from water, PEG and fl-dextran to have a concentration of PEG of 5 massl % and a concentration of fl-dextran of 20 nM.

An assay was performed as describe in connection with FIG. 1.

The prepared mother sample (a) was applied in a sample chamber 7 of the sample compartment 3 and the temperature of the mother sample was set to 50° C. The sample was withdrawn from the mother sample (a) and pumped into the introduction section of the tube in the maintaining compartment, where it was subjected to a 25 degrees temperature jump from 50° C. to 25° C. Fluorescent intensity readings were performed at the read out section as the sample passes through.

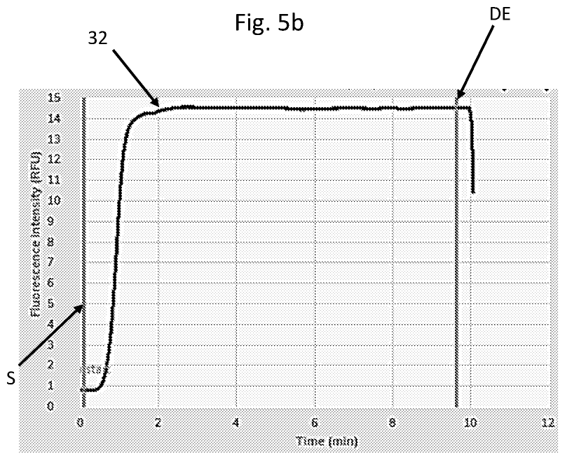

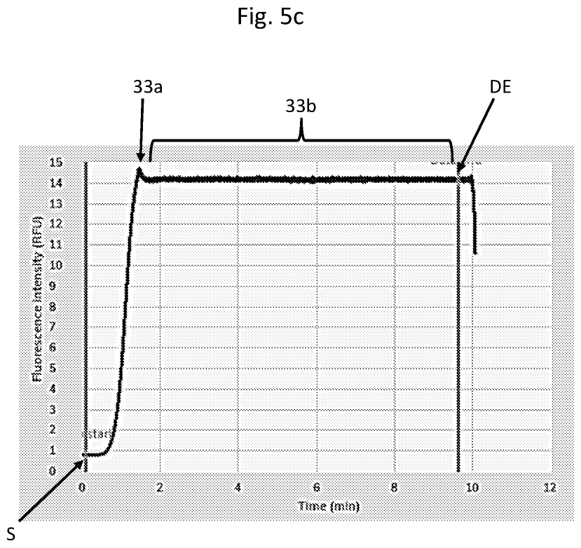

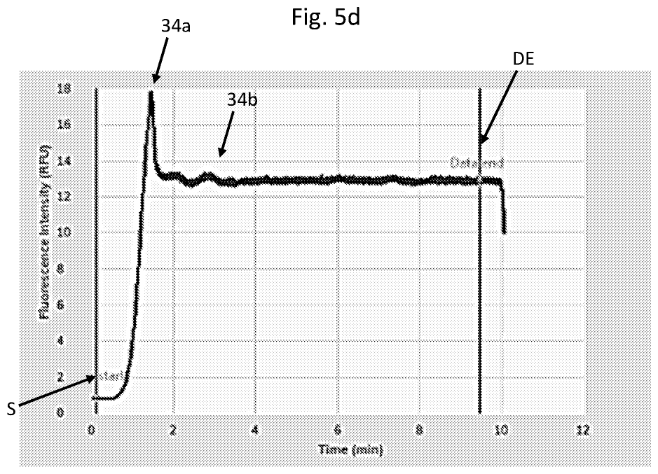

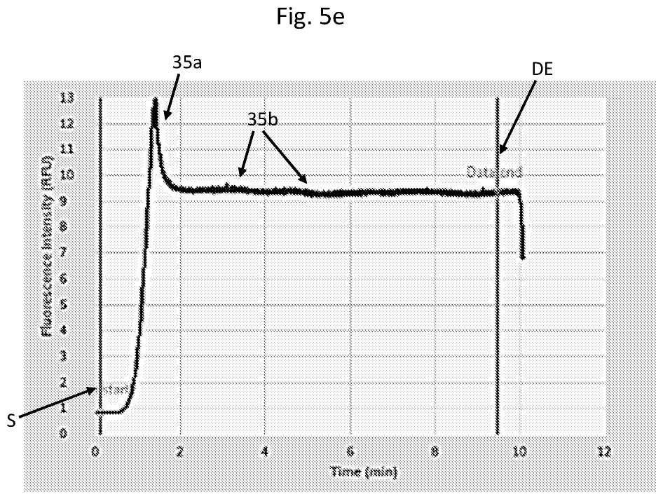

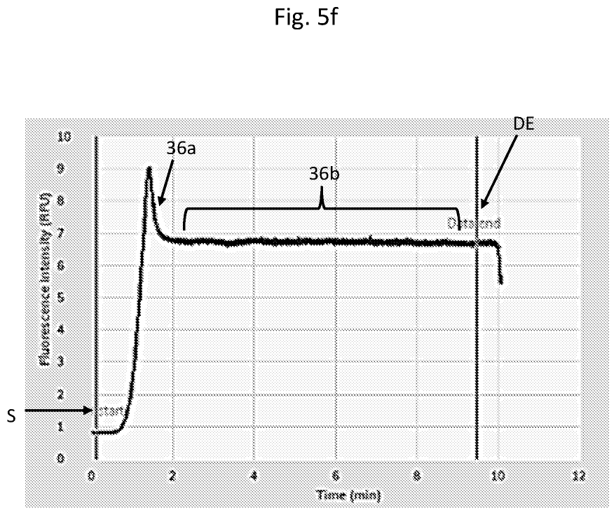

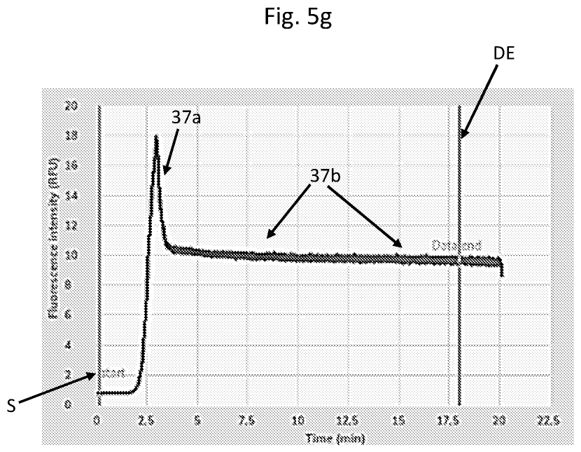

The resulting readings at the read out section are shown in FIG. 5a.

The reference “s” indicates the start of reading. The first few seconds of the readings, the sample has not fully reached the read out section. As the sample reaches the read out section, the signal raises to its maximal level and remains substantially stably during the remaining reading time until data end (DE). From this, it can be concluded that there remains one single phase from start to end of experiment. I.e. no liquid-liquid phase separation takes place.

### Example 2b—LLPS Assay

A mother sample (b) was prepared from the same materials as listed in example 2a.

The aqueous mother sample (b) were prepared from water, Dextran, PEG and fl-dextran to have a concentration of PEG of 5 mass %, a concentration of Dextran of 1 mass % and a concentration of fl-dextran of 20 nM.

The assay was performed as described in example 2a.

The resulting readings at the read out section are shown in FIG. 5b.

The curve obtained in 5b is very similar to the curve of FIG. 5a, however, with a little instability immediately after having reached its maximal level as indicated with ref. 32.

In addition the maximal level reached in FIG. 5b, is slightly lower than the level reached in FIG. 5a.

These characteristic indicates that the single phase of the sample becomes instable and indicates signs of liquid-liquid phase separation e.g. formations of sprinkles or bobbles of a separated phase.

### Example 2c—LLPS Assay

A mother sample (c) was prepared from the same materials as listed in example 2a.

The aqueous mother sample (c) were prepared from water, Dextran, PEG and fl-dextran to have a concentration of PEG of 5 mass %, a concentration of Dextran of 2 mass % and a concentration of fl-dextran of 20 nM.

The assay was performed as described in example 2a.

The resulting readings at the read out section are shown in FIG. 5c.

In the curve obtained in 5c a clear spike is visible immediately after the signal has reached its maximal level as indicated with ref. 33a. After the spike 33a the signal intensity drops to a lower level 33b, which level is also lower than the general max intensity level shown in FIGS. 5a and 5b.

These characteristic indicates that the sample has initiated liquid-liquid phase separation. The instability of the signal intensity at the lower level 33b also indicates formations of sprinkles or bobbles of a separated phase.

### Example 2d—LLPS Assay

A mother sample (d) was prepared from the same materials as listed in example 2a.

The aqueous mother sample (d) were prepared from water, Dextran, PEG and fl-dextran to have a concentration of PEG of 5 mass %, a concentration of Dextran of 3 mass % and a concentration of fl-dextran of 20 nM.

The assay was performed as described in example 2a.

The resulting readings at the read out section are shown in FIG. 5d.

The curve obtained in 5d shows a very significant spike 34a and an increased instability of the intensity level 34b after the spike 34a.

In addition, it can be observed that the intensity level after the spike 34a is generally lower than in the previous LLPS assays with lower amount of Dextran.

These characteristic indicates a clear liquid-liquid phase separation of the sample and that formations of sprinkles or bobbles of a separated phase has taken place.

### Example 2e—LLPS Assay

A mother sample (e) was prepared from the same materials as listed in example 2a.

The aqueous mother sample (e) were prepared from water, Dextran, PEG and fl-dextran to have a concentration of PEG of 5 mass %, a concentration of Dextran of 4 mass % and a concentration of fl-dextran of 20 nM.

The assay was performed as described in example 2a.

The resulting readings at the read out section are shown in FIG. 5e.

The curve obtained in 5e shows a very significant spike 35a. In addition, the intensity level 35b after the spike 35a is significantly lower than in the previous LLPS assays with lower amount of Dextran e.g. as in example 2d/FIG. 5d. Comparing the intensity level 35b after the spike 35a of FIG. 5e with the intensity level 34b after the spike 34a of FIG. 2d, the intensity level in 5e in almost 30% lower.

These characteristic indicates that the formations of sprinkles or bobbles of separated phase is larger in example 2e than in example 2d.

### Example 2f—LLPS Assay

A mother sample (f) was prepared from the same materials as listed in example 2a.

The aqueous mother sample (f) were prepared from water, Dextran, PEG and fl-dextran to have a concentration of PEG of 5 mass %, a concentration of Dextran of 5 mass % and a concentration of fl-dextran of 20 nM.

The assay was performed as described in example 2a.

The resulting readings at the read out section are shown in FIG. 5f.

The curve obtained in 5f shows a very significant spike 36a. In addition, the intensity level 36b after the spike 35a is even lover lower than in example 2e/FIG. 5e. This indicates that the liquid-liquid phase separation is even more pregnant and that larger volume of sprinkles or bobbles of separated phase have been formed.

### Example 2g—LLPS Assay

A mother sample (g) was prepared from the same materials as listed in example 2a.

The aqueous mother sample (f) were prepared from water, Dextran, PEG and fl-HSA to have a concentration of PEG of 5 mass %, a concentration of Dextran of 4 mass % and a concentration of fl-dextran of 50 nM.

The assay was performed as described in example 2a.

The resulting readings at the read out section are shown in FIG. 5g.

The curve obtained in 5g shows a very high and significant spike 37, clearly indicating the liquid-liquid phase separation takes place after a few minutes from the temperature jump. After the spike 37, the intensity level drops about 45% and the intensity signal shows increasingly instability over time, which is a clear indication of formations of sprinkles or bobbles of separated phase.

1-111. (canceled)

112. A method for determining a characteristic property of a molecular interaction, the method comprising:
providing a liquid sample comprising a particle capable of being in a state of equilibrium and in a state of non-equilibrium in the liquid sample, the particle comprises a marker in at least one of its state of equilibrium and state of non-equilibrium;
bringing the particle in a state of non-equilibrium by subjecting the sample to a condition jump;
reading out the marker as a function of time during at least a portion of a relaxation time for the particle; and
determining the characteristic property of the molecular interaction,
wherein the condition jump comprises subjecting the sample to a jump in temperature from at least one first temperature to a second condition at a second temperature and the method further comprises maintaining the second temperature during at least a part of the time of reading out of the marker.

113. The method of claim 112, wherein the reading out comprises performing two or more readings from different fractions of the sample.

114. The method of claim 112, wherein the particle being capable of being in a state of equilibrium and in a state of non-equilibrium:
in that the sample comprises a binding partner for the particle, wherein the liquid sample comprises the particle and the binding partner in chemical equilibrium at the time of initiating the condition jump and wherein at least one of the particle or the binding partner comprises the marker; or
in that the particle has a structure that depends on temperature, wherein the particle has a structure at equilibrium at the second condition, which differs from its structure prior to the condition jump.

115. The method of claim 112, wherein the particle is a protein and the structure difference and/or change is a difference and/or change in at least one folding of the protein.

116. The method of claim 112, wherein the particle has a conformation at equilibrium at the second condition, which differs from its conformation prior to the condition jump.

117. The method of claim 112, wherein the jump in temperature of the sample is performed in the microfluidic unit, the method comprises introducing the sample into the microfluidic unit, wherein the microfluidic unit is at least partly located in a temperature controlled maintaining compartment.

118. The method of claim 117, wherein the microfluidic unit comprises an introduction section to which the sample is introduced, the introduction section comprises a cross-sectional dimension of about 1 mm or less.

119. The method of claim 117, wherein the temperature controlled maintaining compartment is maintained at the second temperature during at least a portion of the relaxation time.

120. The method of claim 117, wherein the temperature controlled maintaining compartment is temperature controlled:
by a method comprising blowing of air;
by a method comprising fully or partly filling the compartment with liquid and/or vapor; or
by a method comprising applying a high voltage to the sample while the sample is located in a container, which forms part of or comprises at least a part of the microfluidic unit.

121. The method of claim 112, wherein the temperature jump from the at least one first temperature to the second temperature comprises providing a temperature jump of at least about 2° C.

122. The method of claim 112, wherein the second temperature is from about 5° C. to about 50° C.

123. The method of claim 112, wherein the microfluidic unit comprises an introduction section and a reading out section, and wherein the reading out comprises performing readings of the sample while the sample is flowing in the microfluidic unit, wherein the reading out as a function of time comprises performing the two or more readings from different fractions of the sample as the sample is flowing in the reading section of the microfluidic unit.

124. The method of claim 112, wherein the reading out as a function of time comprises performing consecutive readings from different fractions of the sample as the respective sample fractions are passing a reading location of the microfluidic unit.

125. The method of claim 112, wherein the method comprises determining at least one of a kinetic parameter, a partitioning parameter, a degradation parameter, an oligomerization parameter, and a folding parameter.

126. The method of claim 112, wherein the molecular interaction comprises a liquid-liquid phase separation, wherein the particle comprises at least two different molecules and an optional additional solvent, which molecules are capable of forming a liquid-liquid phase separation at the condition prior to or after the temperature jump.

127. The method of claim 126, wherein the liquid sample at the time immediately prior to subjecting the sample to the temperature jump is in a single phase condition, and wherein the liquid-liquid phase separation comprises at least local formation of a first liquid phase with an interface to a second liquid phase.

128. The method of claim 127, wherein the first liquid phase and the seconds liquid phase differs from each other with respect to concentration and/or presence of at least one molecule.

129. The method of claim 127, wherein the temperature jump is a jump from a higher temperature to a lower temperature, wherein the sample is in a single-phase condition at the higher temperature.

130. The method of claim 126, wherein the sample is subjected to the temperature jump in the channel of the microfluidic unit and the reading out is performed in the channel, wherein the sample is fed to the channel at a pressure to ensure a selected velocity of the sample in the channel, wherein the velocity is adjustable.

131. An apparatus suitable for determining a characteristic property of a molecular interaction by the method of claim 112, the apparatus comprising:
a sample compartment for containing at least one liquid mother sample;
a withdrawing arrangement arranged for withdrawing a sample from a at least one mother sample stored in the sample compartment
a condition jump arrangement arranged for performing a temperature jump of the sample from at least one first temperature to a second temperature, and
at least one reader arrangement for reading at least one marker as a function of time,
wherein the apparatus further comprises a temperature controlled maintaining compartment for maintaining the sample at the second condition during the reading out of the marker.

132. The apparatus of claim 131, wherein the maintaining compartment comprises a temperature controller arrangement comprising a blower for blowing air at a selected temperature and/or a liquid sprinkler for sprinkling liquid at a selected temperature and/or a liquid filler for fully or partly filling the maintaining compartment with liquid at a selected temperature.

133. An apparatus assembly, comprising the apparatus of claim 131 in combination with the microfluidic unit, wherein the microfluidic unit is at least partly located in the temperature controlled maintaining compartment.
